(19) United States
(12) Patent Application Publication
(10) Pub. No.: US 2023/0108093 A1
(43) Pub. Date: Apr. 6, 2023

(54) METHODS FOR PREDICTING AND TREATING LAMENESS

(71) Applicant: Novus International, Inc., St. Charles, MO (US)

(72) Inventors: Karen Wedekind, St. Charles, MO (US); Heather Tucker, St. Charles, MO (US); Mercedes Vazques-Anon, St. Charles, MO (US); Juxing Chen, St. Charles, MO (US)

(21) Appl. No.: 17/960,534

(22) Filed: Oct. 5, 2022

Related U.S. Application Data
(60) Provisional application No. 63/252,344, filed on Oct. 5, 2021.

(51) Int. Cl. A61K 31/19; G01N 33/68; A61P 19/02

(52) U.S. Cl. CPC A61K 31/19; G01N 33/6887; A61P 19/02; G01N 2800/10

(57) ABSTRACT

The present disclosure provides methods for quantitating the degree of lameness in pigs based on serum biomarker levels. Also provided are methods for treating lameness in pigs induced by fast body weight gain, wherein the methods comprise administering at least one organic mineral. The present disclosure also provides methods for detecting lameness in pigs based upon the levels of saliva biomarkers.

### CROSS-REFERENCE TO RELATED APPLICATIONS

This application claims priority to U.S. Provisional Application No. 63/252,344 filed Oct. 5, 2021, entitled “METHODS FOR PREDICTING AND TREATING LAMENESS”, the entire contents of which are incorporated by reference herein.

### FIELD OF THE INVENTION

The present disclosure relates to methods for diagnosing lameness, quantitating the degree of lameness, and treating lameness in animals.

### BACKGROUND

Lameness has been identified as a welfare issue in all livestock species that leads to reduction in productivity and profitability of the farm. Lameness is a failure of the animal to walk normally. Although lameness can be caused by congenital or developmental abnormalities, most lameness in production animals is caused by pain associated with infections, trauma-related injuries, or dietary causes. Visual gait lameness scores are commonly used to identify lameness in production animals. However, this subjective scoring system lacks sensitivity and consistency among scorers and leads to delayed detection of lameness. Earlier detection of lameness improves the opportunity for resolution with nutritional programs. Thus, there is a need for accurate, objective methodologies that will enable earlier identification of lameness. Furthermore, solutions are needed for reducing severity and incidence of lameness such as nutritional intervention.

### SUMMARY

One aspect of the present disclosure provides methods for quantifying lameness in a pig. The methods comprise (a) determining a level of at least one biomarker and/or a ratio of two biomarkers in serum samples from the pigs, wherein the at least one biomarker is C2C, CTX1, CTX2, OC, or P2CP, and the ratio of two biomarkers is OC/CTX1, P2CP/C2C, or P2CP/CTX2; (b) generating a test score by standardizing the data from (a) to have a mean of 0 and a standard deviation of 1; and (c) using the test score to quantify lameness, wherein increasing deviation of the test score from the mean correlates with increased lameness.

Another aspect of the present disclosure encompasses methods for quantifying lameness in a population of pigs. The methods comprise (a) determining a level of at least one biomarker and/or a ratio of two biomarkers in serum samples from a subset of pigs from the population of pigs, wherein the at least one biomarker is C2C, CTX1, CTX2, OC, or P2CP, and the ratio of two biomarkers is OC/CTX1, P2CP/C2C, or P2CP/CTX2; (b) generating a test score by standardizing the data from (a) to have a mean of 0 and a standard deviation of 1; and (c) using the test score to quantify lameness in the population of pigs, wherein increasing deviation of the test score from the mean correlates with increased lameness.

In some embodiments, an increase or decrease from about 0.1 to about 0.2 units from the mean correlates with a lameness gait score of about 2.0. In some embodiments, an increase or decrease of about 0.3 to about 0.5 units from the mean correlates with a lameness gait score of about 3.0. In still other embodiments, an increase or decrease of about 0.5 units from the mean or greater correlates with a lameness gait score of about 4.0.

In some embodiments, the lameness is due to natural causes or due to fast body weight gain.

In some embodiments, increasingly positive test scores for P2CP, P2CP/C2C, and P2CP/CTX2 correlate with increased lameness. In some additional embodiments, increasingly negative test scores for C2C, CTX1, and OC correlate with increased lameness. In still further embodiments, the relationship between test scores and gait scores is shown in FIG. 1.

Another aspect of the present disclosure encompasses methods for treating lameness in pigs. One method is for treating lameness due to fast body weight gain in pigs, wherein the method comprising administering at least one organic mineral to the pigs, wherein administering the at least one organic mineral increases the serum level of OC, decreases the serum level of P2CP, decreases the serum ratio of P2CP/C2C, decreases the serum ratio of P2CP/CTX2, and/or increases the serum ratio of OC/CTX1 and reduces symptoms of lameness as compared to administering at least one inorganic mineral to comparable pigs afflicted with lameness. Another method for treating lameness due to fast body weight gain in a pig, wherein the method comprises administering at least one metal chelate of methionine hydroxy analog (MHA) to the pig, wherein administering the at least one metal chelate of MHA reduces symptoms of lameness as compared to administering at least one inorganic mineral to a comparable pig afflicted with lameness.

In some embodiments, the at least one organic mineral comprises at least one metal ion bonded to at least one organic ligand to form a chelate, a complex, or a salt. In some aspects, the at least one metal ion is calcium, chromium, cobalt, copper, germanium, iron, lithium, magnesium, manganese, molybdenum, nickel, potassium, rubidium, sodium, tin, vanadium, zinc, or a combination thereof. In a preferred aspect, the at least one metal chelate of MHA comprises a metal ion chosen from copper, manganese, zinc, or a combination thereof. In some aspects, the at least one organic ligand is an amino acid, an amino acid analog, a peptide, a protein, a protein hydrolysate, a hydroxy acid, an organic acid, a sugar alcohol, a polysaccharide, or a polynucleic acid. In some further aspects, the at least one organic mineral is a metal amino acid complex or a metal proteinate. In preferred aspects, the at least one organic mineral is a metal chelate of MHA.

In some embodiments, fast body weight gain is defined as an increased average weight gain of about 50 g/day to about 150 g/day relative to normal growth rate in grower or finisher pigs, or an average weight gain of about 400 g/day to about 500 g/day from mating to farrowing in gestating gilts or sows. In still further embodiments, fast body weight gain is defined an increase in average daily gain of about 7% to about 10% relative to normal growth rate.

In some embodiments, administering the at least one metal chelate of MHA does not reduce the rate of body weight gain in the pig. In some additional embodiments, fast body weight gain is due to an increased level of at least one nutrient in the diet of the pig. In other embodiments, fast body weight gain is due to addition of at least one growth promoter in the diet of the pig. In some aspects, the growth promoter is a beta-adrenergic agonist, an antimicrobial growth promoter, an acidic growth promoter, a mineral clay, an enzyme, a metallic substance, a plant-based nutraceutical, or a prebiotic/probiotic. Preferably, the growth promoter is a beta-adrenergic agonist, such as ractopamine.

Still another aspect of the present disclosure provides methods detecting lameness in a pig, wherein the method comprises (a) determining a level of at least one biomarker and/or a ratio of two biomarkers in a saliva sample from the pig, wherein the at least one biomarker is C2C, CTX1, CTX2, OC, or P2CP, and the ratio of two biomarkers is OC/CTX1, P2CP/C2C or P2CP/CTX2, and (b) comparing the level of the at least one biomarker and/or the ratio of two biomarkers in the saliva sample to a lameness negative reference, wherein a change in the level of the at least one biomarker and/or the ratio of two biomarkers in the saliva sample relative to the lameness negative reference indicates that the pig is lame or is predisposed to becoming lame. In some embodiments, the method further comprises (c) administering at least one organic mineral to the pig that is lame or is predisposed to becoming lame, wherein administering the at least one organic mineral reduces symptoms of lameness as compared to administering at least one inorganic mineral to a comparable pig afflicted with lameness.

In some embodiments, the at least one biomarker is CTX1 or P2CP, and an increased level of CTX1 or P2CP in the saliva sample indicates that the pig is lame or is predisposed to becoming lame. In other embodiments, the ratio of two biomarkers is P2CP/C2C or P2CP/CTX2, and an increased level of P2CP/C2C or P2CP/CTX2 in the saliva sample indicates that the pig is lame or is predisposed to becoming lame.

In some embodiments, the at least one organic mineral comprises at least one metal ion bonded to at least one organic ligand to form a chelate, a complex, or a salt. In some aspects, the at least one metal ion is calcium, chromium, cobalt, copper, germanium, iron, lithium, magnesium, manganese, molybdenum, nickel, potassium, rubidium, sodium, tin, vanadium, zinc, or a combination thereof. In a preferred aspect, the at least one metal ion of the at least one organic mineral is copper, manganese, zinc, or a combination thereof. In some additional aspects, the at least one organic ligand is an amino acid, an amino acid analog, a peptide, a protein, a protein hydrolysate, a hydroxy acid, an organic acid, a sugar alcohol, a polysaccharide, or a polynucleic acid. In still further aspects, the at least one organic mineral is a metal amino acid complex or a metal proteinate. In other aspects, the at least one organic mineral is a metal chelate of MHA.

In some embodiments, the lameness is due to a dietary deficiency.

Other aspects and iterations of the disclosure are described in more detail below.

### BRIEF DESCRIPTION OF THE DRAWINGS

FIG. 1 presents the relationship between serum biomarker levels and gait score in pigs. The average standardized biomarkers are plotted as a function of gait score.

FIGS. 2A-2C show results of a fast growth study in pigs. Diets were supplemented with inorganic trace minerals (ITM) or metal chelate of MHA (MTX). Fast growth was induced by including ractopamine (RAC) in the diet. FIG. 2A presents the average daily growth (ADG) of the pigs in the study and FIG. 2B presents the gait score on Day 70. FIG. 2C presents the increase in bone ash measured at the end of the trial in a subset of pigs (n=53).

FIG. 3 shows results of a lameness study in pigs with diets supplemented with inorganic trace minerals (ITM) and various organic trace minerals (Mintrex, OTM Competitor #1, OTM Competitor #2).

FIGS. 4A and 4B show the distribution of lameness in pigs fed diets supplemented with inorganic trace minerals (ITM) or Mintrex in two separate trials. FIGS. 4C and 4D show the difference in average daily growth between healthy and lame pigs in two separate trials. The x-axis represents two-week intervals.

FIG. 5 shows average mean osteocalcin concentrations in serum in pigs following supplementation with inorganic trace minerals (ITM), OTM Competitor #1, OTM Competitor #2, or Mintrex (MTX).

FIG. 6 shows average P2CP concentrations in serum in pigs following supplementation with inorganic trace minerals (ITM), OTM Competitor #1, OTM Competitor #2 or Mintrex (MTX).

FIG. 7 shows average P2CP concentrations in saliva in pigs following supplementation with inorganic trace minerals (ITM), OTM Competitor #1, OTM Competitor #2 or Mintrex (MTX).

FIG. 8 shows a relationship between biomarkers and gait score in a population with infectious inflammatory, naturally occurring chronic lameness.

FIGS. 9A-9G shows a relationship between C2C (9A), CTX1 (9B), Ostec (9C), P2CP (9D), the ratio of P2CP/C2C (9E), the ratio of P2CP/CTX2 (9F), and CTX2 (9G) and gait score in 5 trial dataset. Datapoints circled are significantly different from 0.

### DETAILED DESCRIPTION

The present disclosure provides methods for quantitating lameness in pigs based on serum biomarker levels, methods for treating lameness, and methods for detecting lameness based on saliva biomarker levels.

### (I) Lameness and Biomarkers

Traditionally, lameness in animals has been diagnosed and/or monitored visually. For example, visual gait scoring systems have been developed to assign gait scores based on observable features of lameness. More recently, biomarkers have been identified that are associated with lameness. The relationship between gait scores and biomarkers has not been defined in the literature. Provided herein are methods for more accurately quantifying lameness with more precise biomarker measurements. Such measurements reflect changes in the skeleton health of the animals non-invasively and provide more information than the absence/presence of a bone disorder.
(a) Lameness

Lameness is the inability of a pig to walk normally. Mildly affected pigs may have a disturbed gait with swaying of the hips and/or a shortened stride, while more severely affected pigs may be reluctant to stand or move. In general, lameness in production pigs and breeding stock may be due to infections, physical or trauma-related injuries, or dietary issues (e.g., nutritional deficiencies). In many instances, however, no obvious cause of lameness can be identified, and the lameness may be described as naturally occurring or due to natural causes (which may include congenital or developmental abnormalities and/or underlying metabolic disorders).

Infections that may lead to lameness include brucellosis, clostridial diseases, erysipelas, foot-and-mouth disease, foot rot, bush foot, Glässers disease (e.g., Haemophilus parasuis), mycoplasma infection (e.g., Mycoplasma hyosynoviae, Mycoplasma hyorhinis), salmonellosis, swine vesicular disease, and streptococcal infections. Infections can also occur due to bacteria entering a wound after a physical injury resulting in swollen joints and abscesses.

Physical or trauma-related injuries can result from inappropriate or poorly maintained floors in the pig facility. Injuries include wounds directly to the foot or damage to the joints, ligaments, or muscles caused by pigs slipping on the floor surface, as well as fighting and/or mounting behavior among pigs.

Dietary causes of lameness may include diets deficient in phosphorus, calcium, and/or vitamin D, which can lead to bone metabolic diseases or disorders. Such dietary deficiencies may be inadvertent in that the levels of phosphorus, calcium, phytase, and/or vitamin D are not at the expected levels in the products included in the diet because of storage, processing, or other issues.

Diets that are designed to increase growth rate or body weight gain also may lead to lameness. For example, the increased muscle mass places additional stress on the joints and bones, which may lead to lameness. Fast growth or fast body weight gain in grower or finisher pigs may be defined as an increased average weight gain of about 50 g/day to about 150 g/day (e.g., about 100 g/day) relative to normal growth rate. Fast growth or fast weight gain in gestating gilts or sows may be defined as an average weight gain about 400 g/d to about 500 g/d during from mating to farrowing. In still other embodiments, fast growth or fast weight gain may be defined as an increase in average daily gain (ADG) of about 6% to about 12% (e.g., or about 7% to about 10%) relative to normally growing pigs.

Fast growth or body weight gain may be induced by diets containing increased levels of nutrients as compared to the standardized pig diet. Increased dietary protein, amino acids, etc. and/or increased feed energy density typically supports higher body weight gains. Nutrients include proteins, amino acids, fats, lipids, and saccharides (e.g., molasses, corn syrup, rice bran syrup, etc.) Fast growth or fast body weight gain also may be induced by the addition of at least one growth promoter in the diet of the pig. Growth promoters include antimicrobial growth promoters (e.g., antibiotics), acidic growth promoters (e.g., organic acids such as formic, acetic, propionic, citric, fumaric, malic acids), mineral clays (e.g., attapulgite which can absorb toxins from the digestive system), enzymes (e.g., beta-glucanase, xylanase, proteases, etc.), metallic substances, (e.g., copper sulfate, zinc oxide, etc.), plant-based nutraceuticals (e.g., essential oils and other agents extracted from garlic, ginseng, oregano, cinnamon, aniseed, rosemary, peppermint, etc.), and/or prebiotic/probiotics (e.g., agents derived from yeast or bacteria that promote good digestive health). Growth promoting antibiotics include avilamycin, carbadox, flavomycin, olaquindox, spiramycin, salinomycin, tylosin, virginiamycin, and bacitracin.

In particular, fast growth may be induced by diets containing a beta-adrenergic agonist such as ractopamine. Ractopamine is a phenethanolamine-repartitioning agent that reduces adipose and increases lean deposition resulting in increased growth performance, carcass leanness and dressing percentage. Other beta-adrenergic agonists that may result in fast growth include brombuterol, cimaterol, clenbuterol, clenproperol, isoxsuprine, mabuterol, salbutamol, and terbutaline.

Lameness may be monitored visually using subjective gait scoring systems. The gait or locomotion of animals is observed and scored to give a subjective measurement of lameness. For example, a five-point gait score system is detailed below.

| Lameness | |
|---|---|
| Score | Description |
| 0 | Normal gait. Animal moves freely and used all four limbs and |
| | feet evenly. |
| 1 | Uneven gait. Animal shows weight-shifting activities away |
| | from affected limb upon standing but show no lameness of |
| | limping when walking. |
| 2 | Mildly lame. Animal obviously shifts weight away from |
| | affected limb when standing and shows limping or adaptive |
| | behavior when walking (e.g., head bob, quickened step on |
| | affected limb). |
| 3 | Moderately lame. Animal is reluctant to stand and/or walk, |
| | obvious limp and adaptive behaviors when walking. |
| 4 | Severely lame. Animal is non-weight bearing on the affected |
| | limb when either standing or walking, very reluctant to move. |

When administered by properly trained individuals, visual gait scoring evaluations can be reliable and repeatable, especially for noticeably lame animals. However, identifying mildly lame animals with visual gait scoring systems is much more challenging.

Lameness may also be assessed using force plate tests, which measure the amount of force each limb applies to separate loading cells. A lame animal typically bears less weight on the limb that is painful or structurally unsound. Force plate tests, however, tend to be quite time- and labor-intensive.
(b) Biomarkers

Biomarkers are measurable substances whose presence in an organism is indicative of some phenomenon such as disease, infection, or environmental exposure. Bone and cartilage biomarkers that are associated with lameness have been validated in a variety of animals. The levels of bone and cartilage biomarkers may increase or decrease in lame animals as compared to sound animals. Moreover, the levels of bone and cartilage biomarkers may change differently depending upon the cause of lameness.

A bone synthesis biomarker is osteocalcin (OC). Also known as bone gamma-carboxyglutamic acid-containing protein (BGLAP). Osteocalcin is the most abundant non-collagenous protein in bone and is specifically expressed in osteoblasts.

A bone degradation biomarker is C-terminal telopeptide of type I collagen (CTX1, CTX-1, or CTX-I), which is released by cathepsin K cleavage of intact bone type 1 collagen. Type I collagen accounts for about 90% of the organic matrix of bone. CTX1 relates to bone turnover because it is the portion of the molecule that is cleaved by osteoclasts during bone resorption.

A cartilage synthesis biomarker is procollagen type II C-terminal propeptide or P2CP (also known as C-propeptide if type II collagen or CPII). P2CP is a product of collagen II synthesis. Type II collagen is the major organic constituent of cartilage.

A cartilage degradation biomarker is C-terminal telopeptide of type II collagen (CTX2, CTX-2, or CTX-II). Following the degradation of cartilage, fragments of CTX-2 are released into circulation.

Yet another cartilage degradation biomarker is collagen type II cleavage or C2C. Type II collagen, the major organic constituent of cartilage, is cleaved into fragments by collagenases.

Ratios of synthesis to degradation biomarkers are also indicators of bone health or cartilage health. For example, the ratios of OC/CTX1, P2CP/C2C, P2CP/CTX2 may be used as indicators of lameness.

Non-limiting example of additional biomarkers that represent bone and cartilage metabolism include inflammatory biomarkers such as adenosine deaminase (ADA), C-reactive protein (C-RP), hyaluronic acid, adipsin, leptin, TNF-alpha, IL-1beta, and IL-6; cartilage catabolism markers such as matrix metalloproteinase-1, 3, 9 (MMP-1, MMP-3, MMP-9), chondroitin sulfate epitope 846 (CS846), and glycosaminoglycan (GAG); bone degradative biomarkers such as pyridinoline (Pyd); and bone synthesis biomarkers such as bone specific alkaline phosphatase (BAP).

### (II) Methods for Quantifying Lameness

One aspect of the present disclosure provides methods for quantifying the severity or lameness in pigs based on changes in the levels and/or ratios of bone and cartilage biomarkers in serum samples. The methods disclosed herein may be used to quantitate lameness due to natural cases, infections, injuries, or dietary issues. In some embodiments the methods may quantitate lameness due to natural causes. On other embodiments, the methods may quantitate lameness due to fast growth or fast body weight gain. The methods for quantifying lameness comprise (a) determining the level of at least one biomarker and/or the ratio of two biomarkers in a serum sample from the pig; (b) generating a test score by standardizing the data from (a) to have a mean of 0 and a standard deviation of 1; and (c) using the test score to quantify lameness, wherein increasing deviation of the test score from the mean correlates with increased lameness.

In some embodiments, the methods comprise quantitating lameness in a population of pigs, wherein the levels and/or ratios of the biomarkers are determined in a subset of pigs from the population of pigs such that an average degree of lameness can be estimated in the population of pigs.

Lameness and its causes are described above in section (I)(a). Examples of suitable biomarkers are described above in section (I)(b).

Lameness may be quantified in pigs of different ages, sexes, and/or breeds or genetics. For example, the pig may be a piglet or shoat, a starter pig, a grower pig, a finisher pig, a breeder pig, a gilt, a sow, a barrow, a hog, or a boar. In general, the pig is a commercial pig breed. Non-limiting examples of commercial pig breeds include Berkshire, Chester white, Duroc, Hampshire, Landrace. Poland China, Spotted, Yorkshire, Meishan, Fengiing, Jiaxing black, Erhualian, and Pietrain, and non-limiting examples of commercially available pig genetics include PIC, Danbred, Topigs, and Hypor.
(a) Determining Serum Biomarker Levels and/or Ratios

The first step of the method comprises determining the levels and/or ratios of bone and or cartilage biomarkers in a serum sample from the pig. To accomplish this, first a blood sample is collected from the pig. Various methods of collecting blood are known in the art. Generally, a method of collecting blood comprises accessing the blood using a skin-piercing element and collecting the blood therein into some type of a collection device. Accessing the blood may also involve the use of a fluid pathway, a capillary channel (e.g., a capillary tube), a fluid transfer medium (e.g., a hydrophilic porous material), or some kind of mechanical or vacuum means in conjunction with the skin-piercing element. The blood is generally transferred to a tube and the blood is allowed to clot (e.g., 15-30 min at room temperature). The tube of clotted blood may be centrifuged (e.g., 1000-2000×g for about 10 min) to collect the blood cells and clotting factors in the bottom of the tube. The resulting supernatant is the serum, which may be transferred to a new tube and stored at the appropriate temperature (e.g., 4° C., −20° C., or −70° C.) until further analysis.

A variety of techniques may be used to determine the level or concentration of the at least one biomarker of interest. The biomarker may be detected and quantified using an antibody-based detection method. For example, the level of the biomarker may be determined using an enzyme-linked immunosorbent assay (ELISA). The ELISA may be a direct ELISA, a sandwich ELISA, a competitive ELISA, or a reverse ELISA. The detection method may be optical (e.g., colorimetric or fluorometric) or electrochemical. In specific embodiments, the biomarker(s) may be detected using a sandwich ELISA with colorimetric detection.

On other embodiments, the antibody-based detection method may comprise protein immunoprecipitation, immunoelectrophoresis, Western blotting, or protein immunostaining. In still other embodiments, the biomarker(s) levels may be quantitated using high performance liquid chromatography (HPLC) or liquid chromatography—mass spectrometry (LC/MS).

In specific embodiments, the at least one biomarker of interest may be chosen from C2C, CTX1, CTX2, OC, and P2CP. In some embodiments, the level of one biomarker is determined. In other embodiments, the levels of two biomarkers are determined. In still other embodiments, the levels of three biomarkers are determined. In further embodiments, the levels of four biomarkers are determined. In additional embodiments, the levels of five biomarkers are determined.

Once the levels of certain biomarkers are determined, ratios of synthesis to degradation biomarkers can be determined.
(b) Generating a Test Score

The next step of the method comprises generating a test score by standardizing the levels and/or ratios of the measured biomarkers. Standardization is used because of differences in the ranges of the biomarkers, differences among the various detection methods, etc. The data are standardized to have a mean of 0 and a standard deviation of 1. A variety of methods are available for data standardization. For example, various statistical analysis packages (e.g., SAS) are commercially available.
(c) Quantifying Lameness

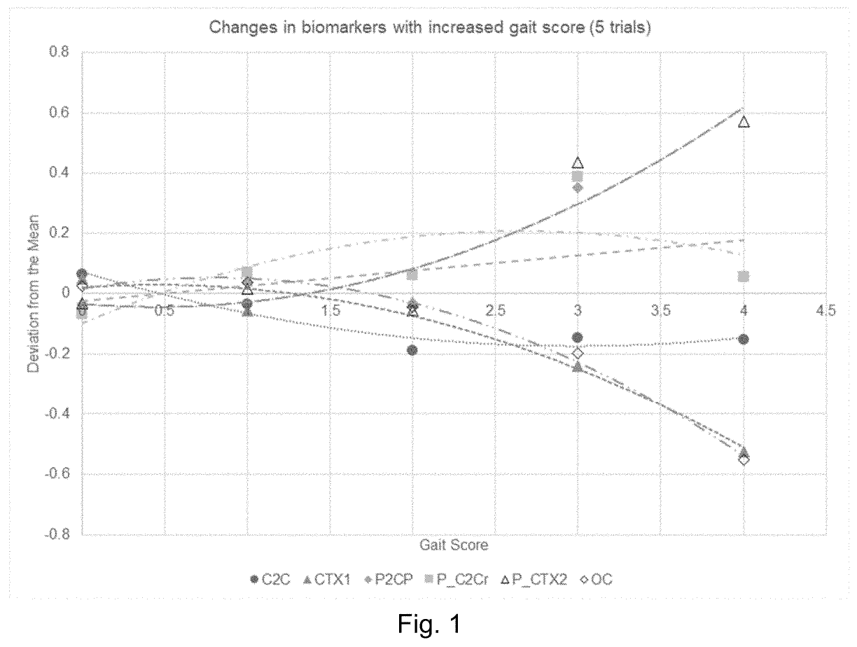

The final step of the method comprises using the test score to quantify lameness in the pig, wherein increasing deviation of the test score from the mean correlates with increased lameness. FIG. 1 presents the relationship between biomarker standardized test scores and gait scores. There is little variation of the standardized test scores at gait scores less than about 2.0 and there is increasing variation of the standardized test scores at gait scores greater than about 2.0. Thus, increasing deviation of the standardized test scores from the mean correlates with increased lameness.

In some embodiments, an increase or decrease of about 0.1 to about 0.3 units from the mean correlates with a lameness gait score of about 2.0; for example, a change of about −0.3, −0.2, about −0.1, about 0.1, 0.2, or about 0.3 units from the mean correlates with a gait score of about 2.0. In some aspects, an increase or decrease of about 0.1 to about 0.15, about 0.1 to about 0.2, about 0.1 to about 0.25, about 0.1 to about 0.3, about 0.15 to about 0.3, about 0.2 to about 0.3, or about 0.25 to about 0.3 units from the mean correlates with a gait score of about 2.0.

In other embodiments, an increase or decrease of about 0.3 to about 0.5 units from the mean correlates with a lameness gait score of about 3.0; for example, a change of about −0.5, about −0.4, about −0.3, about 0.3, about 0.4, or about 0.5 units from the mean correlates with a gait score of about 3.0. In some aspects, an increase or decrease of about 0.3 to about 0.35, about 0.3 to about 0.4, about 0.3 to about 0.45, about 0.3 to about 0.5, about 0.35 to about 0.5, about 0.4 to about 0.5, or about 0.45 to about 0.5 units from the mean correlates with a gait score of about 3.0.

In further embodiments, an increase or decrease of about 0.5 units or greater from the mean correlates with a lameness gait score of about 4.0; for example, a change of about -0.5, about -0.6, about -0.7, about -0.8, about 0.5, about 0.6, about 0.7, or about 0.8 units from the mean correlates with a gait score of about 4.0. In some aspects, an increase or decrease of about 0.5 units or greater, about 0.6 units or greater, about 0.7 units or greater, or about 0.8 units or greater correlates with a gait score of about 4.0. In still other aspects, an increase or decrease of about 0.5 to about 0.55, about 0.5 to about 0.6, about 0.5 to about 0.65, about 0.5 to about 0.7, about 0.5 to about 0.75, about 0.5 to about 0.8, about 0.55 to about 0.8, about 0.6 to about 0.8, about 0.65 to about 0.8, about 0.7 to about 0.8, or about 0.75 to about 0.8 correlates with a gait score of about 4.0.

In some embodiments, the at least one biomarker may be C2C. In other embodiments, the at least one biomarker may be CTX1. In further embodiments, the at least one biomarker may be P2CP. In still other embodiments, the at least one biomarker may be C2C and CTX1. In some embodiments, the at least one biomarker may be C2C and P2CP, and the ratio of biomarkers may be P2CP/C2C. In other embodiments, the at least one biomarker may be CTX2 and P2CP, and the ratio of biomarkers may be P2CP/CTX2. In some embodiments, the at least one biomarker may be C2C, CTX1, and P2CP, and the ratio of biomarkers may be P2CP/C2C. In other embodiments, the at least one biomarker may be CTX1, CTX2, and P2CP, and the ratio of biomarkers may be P2CP/CTX2. In some embodiments, the at least one biomarker may be C2C, CTX2, and P2CP, and the ratio of biomarkers may be P2CP/C2C and P2CP/CTX2. In other embodiments, the at least one biomarker may be C2C, CTX1, CTX2, and P2CP, and the ratio of biomarkers may be P2CP/C2C and P2CP/CTX2.

In some embodiments, positive test scores for P2CP, P2CP/C2C, and P2CP/CTX2 correlate with lameness, with increasingly positive test scores correlating with increased severity of lameness. In other embodiments, negative test scores for C2C and CTX1 correlate with lameness, with increasingly negative test scores correlating with increased severity of lameness.

### (III) Methods for Treating Lameness

Another aspect of the present disclosure provides methods for treating lameness, wherein the method comprises administering at least one organic mineral to a pig that is lame or is predisposed to becoming lame, wherein administering the at least one organic mineral reduces symptoms of lameness as compared to administering at least one inorganic mineral to a comparable pig that is lame or predisposed to becoming lame. In specific embodiments, the at least one organic mineral is at least one metal chelate of methionine hydroxy analog (MHA). The lameness that is treated may be due to natural cases, infections, trauma-related injuries, or dietary issues, as described above in section (I)(a).

Also provided herein are methods for treating lameness induced by fast body weight gain. One method comprises administering at least one organic mineral to a pig afflicted with lameness induced by fast body weight gain, wherein administering the at least one organic mineral changes the serum level of at least one biomarker chosen from C2C, CTX1, CTX2, OC, and P2CP and/or the serum ratio of two biomarkers chosen from OC/CTX1, P2CP/C2C, and P2CP/CTX2, and reduces symptoms of lameness as compared to administering at least one inorganic mineral to a comparable pig afflicted with lameness induced by fast body weight gain.

Another method for treating lameness induced by fast body weight gain comprises administering at least one metal chelate of MHA to the pig, wherein administering the at least one metal chelate of MHA reduces symptoms of lameness as compared to administering at least one inorganic mineral to a comparable pig afflicted with lameness induced by fast body weight gain. Advantageously, administering metal chelates of MHA does not reduce the rate of body weight gain in the fast-growing or fast weight-gaining pigs.

In some embodiments, the fast growth or fast body weight gain of the pig is facilitated by including at least one growth promoter and/or including higher levels of protein, amino acids, and/or energy in the diet of the pig, as described above in section (I)(a). In some embodiments, the lameness induced by fast body weight gain is chronic, wherein chronic is defined as the lameness being present for at least one week.

The methods disclosed herein may be used to treat lameness in pigs of different ages, sexes, and/or breeds or genetics. For example, the pig may be a piglet or shoat, a starter pig, a grower pig, a finisher pig, a breeder pig, a gilt, a sow, a barrow, a hog, or a boar. In general, the pig is a commercial pig breed. Non-limiting examples of commercial pig breeds include Berkshire, Chester white, Duroc, Hampshire, Landrace. Poland China, Spotted, Yorkshire, Meishan, Fengiing, Jiaxing black, Erhualian, and Pietrain, and non-limiting examples of commercially available pig genetics include PIC, Danbred, Topigs, and Hypor.
(a) Administering at Least One Organic Mineral

The methods comprise administering at least one organic mineral. An organic mineral comprises at least one metal ion bonded to at least one organic ligand to form a chelate, a complex, or a salt. The at least one metal ion may be a transition metal or a main group metal. The at least one organic ligand may be an amino acid, an amino acid analog, a peptide, a protein, a protein hydrolysate, a hydroxy acid, an organic acid, a sugar alcohol, a polysaccharide, or a polynucleic acid. The at least one metal ion may be bonded to the at least one organic ligand via an ionic bond, a covalent bond, a coordinate bond, a hydrogen bond, a van der Waals interaction, or a combination thereof.

The at least one metal ion may be a transition metal chosen from chromium, cobalt, copper, iron, manganese, molybdenum, nickel, or zinc. The at least one metal ion may be a main group metal chosen from calcium, germanium, lithium, magnesium, potassium, sodium, rubidium, tin, or vanadium. In certain embodiments, the at least one metal ion may be calcium, chromium, cobalt, copper, iron, magnesium, manganese, nickel, potassium, sodium, zinc, or combination thereof. In specific embodiments, the at least one metal ion may be copper, manganese, zinc, or combination thereof.

In some embodiments, the organic ligand may be an amino acid or an amino acid analog. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard amino acids (e.g., L-DOPA, GABA, 2-aminobutyric acid, and the like), amino acid analogs, or combinations thereof. Amino acid analogs include a-hydroxy analogs (e.g., methionine hydroxy analog), as well side chain-protected analogs or N-derivatized amino acids.

In some embodiments, the organic ligand may be a peptide, a protein, or a protein hydrolysate (e.g., partially hydrolyzed protein). The peptide, protein, or protein hydrolysate may be derived from feed grains (e.g., corn, soy, wheat, rice, etc.), other food crops (e.g., legumes, potatoes, sugar beets, etc.), dairy by-products (e.g., whey, casein, etc.), animal by-products (e.g., feather meal, fish meal, etc.), yeast, bacteria. and so forth.

In other embodiments, the organic ligand may be a hydroxy acid. Non-limiting examples of alpha hydroxy acids include citric acid, glycolic acid, lactic acid, malic acid, and tartaric acid. Beta hydroxy acids include without limit beta hydroxybutanoic acid, salicylic acid, tropic acid, and trethocanoic acid.

In further embodiments, the organic ligand may be an organic acid. Non-limiting examples of organic acids include adipic acid, ascorbic acid, caprylic acid, citric acid, fulvic acid, furmaric acid, glucoheptonic acid, gluconic acid, glutaric acid, glycerophosphoric acid, humic acid, lactic acid, ketoglutaric acid, malic acid, malonic acid, orotic acid, oxlic acid, pantothenic acid, picolinic acid, pidolic acid, sebacic acid, succinic acid, and tartaric acid.

In still other embodiments, the organic ligand may be a sugar alcohol. Suitable sugar alcohols include, without limit, sorbitol, mannitol, xylitol, lactitol, isomalt, maltitol, erythritol, and hydrogenated starch hydrolysates (HSH).

In further embodiments, the organic ligand may be a polysaccharide. Non-limiting examples of suitable polysaccharides include cellulose, starch, alginate, pectin, mucopolysaccharides, or carrageenan.

In some embodiments, the at least one organic mineral may be a metal amino complex comprising a metal ion complexed with methionine, lysine, or glycine, thereby forming a metal methionate, metal lysinate, or metal glycinate, respectively. In other embodiments, the at least one organic mineral may be a metal proteinate comprising a metal ion complexed with a protein or protein hydrolysate. The metal ion of the metal amino acid complexes or metal proteinates may be Zn, Cu, or Mn. Such metal amino acid complexes and metal proteinates comprise one metal ion complexed with one organic ligand, wherein such complexes are charged (e.g., have a positive charge).

In specific embodiments, the at least one organic mineral is a metal chelate of MHA (also called 2-hydroxy-4-(methylthio)butanoate or HMTBA). Preferred metal ions include Zn, Cu, and Mn. Such metal chelates of MHA are bis-chelates comprising one metal ion bonded to two molecules of MHA. As such, the metal chelates of MHA are neutral molecules.

In particular embodiments, the metal chelate of MHA may be methionine hydroxy analog copper (i.e., MHA-Cu or (HMTBA)₂-Cu), methionine hydroxy analog manganese (i.e., MHA-Mn or (HMTBA)₂-Mn), methionine hydroxy analog zinc (i.e., MHA-Zn or (HMTBA)₂-Zn), or a combination of any or all of the foregoing (which are available from Novus International, Inc., under the tradename MINTREX®).

Administering at least one metal chelate of MHA in conjunction with the diet that induces fast growth or fast body weight gain does not reduce the rate of body weight gain in the pig, as described above in section (I)(a). Thus, pigs administered metal chelates of MHA may keep gaining weight at same rate as fast-growing or fast body weight-gaining pigs not administered metal chelates of MHA. In some embodiments, pigs administered metal chelates of MHA may keep gaining weight at faster rates than comparable pigs administered metal amino acid complexes or metal proteinates.

Inorganic minerals include metal sulfates, metal oxides, metal hydroxides, metal oxychlorides, metal carbonates, and metal halides, wherein the metal is chosen from calcium, chromium, cobalt, copper, germanium, iron, lithium, magnesium, manganese, molybdenum, nickel, potassium, rubidium, sodium, tin, vanadium, or zinc. In some embodiments, the at least one inorganic mineral may be a metal sulfate.

In general, the organic mineral (or inorganic mineral) is included in the feed rations of the pig. Swine nutrient guidelines are readily available that detail the recommended levels of minerals and other nutrients to be included in the pigs' diet. Additionally, producers of organic minerals generally provide recommendations regarding the amount to be administered to pigs of various ages and/or weights.
(b) Reducing Symptoms of Lameness and Biomarker Changes

Administering the at least one organic mineral reduces symptoms of lameness as compared to administering at least one inorganic mineral to a comparable pig afflicted with lameness induced by fast body weight gain. Reduced symptoms of lameness include less noticeable limping, fewer compensatory behaviors (e.g., dipping of head, raised back, etc.), a more normal gait, walking on all four limbs, less reluctant to stand, easier to walk or move, etc.

Administering the at least one organic mineral also changes the levels and/or ratios of at least one biomarker in the pig. Suitable biomarkers are described above in section (I)(b). Determining the level and/or ratio of the at least one biomarker in a serum sample from the pig is described above in section (II)(a).

In some embodiments, administering the at least one organic mineral significantly increases the serum level of OC, decreases the serum level of P2CP, decreases the serum ratio of P2CP/C2C, decreases the serum ratio of P2CP/CTX2, and/or increases the serum ratio of OC/CTX1, whereas administering at least one inorganic mineral to a comparable pig decreases or results in no significant change in the serum level of OC, increases or results in no significant change in the serum level of P2CP, increases or results in no significant change in the serum ratio of P2CP/C2C, increases or results in no significant change in the serum ratio of P2CP/CTX2, and/or decreases or has no effect on the serum ratio of OC/CTX1.

In specific embodiments, administering the at least one chelate of MHA significantly increases the serum level of OC, decreases the serum level of P2CP, decreases the serum ratio of P2CP/C2C, decreases the serum ratio of P2CP/CTX2, and/or increases the serum ratio of OC/CTX1, whereas administering at least one inorganic mineral to a comparable pig decreases or results in no significant change in the serum level of OC, increases or results in no significant change in the serum level of P2CP, increases or results in no significant change in the serum ratio of P2CP/C2C, increases or results in no significant change in the serum ratio of P2CP/CTX2, and/or decreases or has no effect on the serum ratio of OC/CTX1.

### (IV) Methods for Detecting Lameness—Saliva Biomarkers

A further aspect of the present disclosure provides methods for detecting lameness in pigs by measuring the levels of bone and/or cartilage biomarkers in the saliva of pigs. Detecting lameness by measuring biomarkers in saliva has significant advantages over measuring biomarkers in serum. In particular, it is easier to collect saliva from an animal; therefore, no trained technicians are necessary. Moreover, it is safer for the animal, which does not need to be snared or otherwise captured in order to collect the saliva. The methods comprise (a) determining a level of at least one biomarker and/or a ratio of two biomarkers in a saliva sample from the pig, and (b) comparing the level of the at least one biomarker and/or the ratio of two biomarkers in the saliva sample to a lameness negative reference, wherein a change in the level of the at least one biomarker and/or the ratio of two biomarkers in the saliva sample relative to the lameness negative reference indicates that the pig is lame or is predisposed to becoming lame. The methods may further comprise (c) administering at least one organic mineral to the pig that is lame or is predisposed to becoming lame, wherein administering the at least one organic mineral reduces symptoms of lameness as compared to administering at least one inorganic mineral to a comparable pig afflicted with lameness.

The pig may be a piglet or shoat, a starter pig, a grower pig, a finisher pig, a breeder pig, a gilt, a sow, a barrow, a hog, or a boar. In general, the pig is a commercial pig breed. Non-limiting examples of commercial pig breeds include Berkshire, Chester white, Duroc, Hampshire, Landrace. Poland China, Spotted, Yorkshire, Meishan, Fengiing, Jiaxing black, Erhualian, and Pietrain, and non-limiting examples of commercially available pig genetics include PIC, Danbred, Topigs, and Hypor.

Lameness may be caused by a variety of factors as described above in section (I)(a). In some embodiments, the lameness may be due to a dietary deficiency such as, for example, a phosphorus deficiency.

Suitable bone and cartilage biomarkers are detailed above in section (I)(b).
(a) Determining saliva biomarker levels and/or ratios

The first step of the methods comprises determining the levels and/or ratios of bone and/or cartilage biomarkers in saliva from the pigs. To accomplish this, first saliva samples are collected from the pigs. Various methods of collecting saliva are known in the art. In general, saliva is collected using a suitable collection device. In some embodiments, the collection device may be absorbent (e.g., cotton swabs, cotton rope, absorbent pads, salivettes, sponges, other swab devices, etc.). In other embodiments, the collection device may be a plastic pipette, a straw-like device, a syringe-like device, or a suction device. After collection, the saliva may be recovered from absorbent collection devices by centrifugation. After collection and/or recovery, the saliva may be transferred to an appropriate tube and stored at 4° C., -20° C., or -70° C. until further analysis.

Means for detecting biomarkers are described above in section (II)(a). The levels of the at least one biomarker and/or the ratio of two biomarkers in the saliva sample may be standardized at described above in section (II)(b).
(b) Comparing Saliva Biomarkers Levels to a Reference

The methods further comprise comparing the level of the at least one biomarker and/or the ratio of two biomarkers in the saliva sample to a lameness negative reference, wherein a change in the level of the at least one biomarker and/or the ratio of two biomarkers in the saliva sample relative to the lameness reference indicates that the pig is lame or is predisposed to becoming lame. Surprisingly, it was found that the saliva biomarker levels accurately reflected both bone and cartilage biomarkers measured in serum.

The level of the at least one biomarker and/or the ratio of two biomarkers in the saliva sample may increase or decrease relative to the lameness negative reference. The magnitude of the change from the lameness negative reference may indicate the severity of lameness and/or likelihood of becoming lame.

In some embodiments, an increased level of CTX1 in the saliva sample indicates that the pig is lame or is predisposed to becoming lame. In some additional embodiments, an increased level of P2CP in the saliva sample indicates that the pig is lame or is predisposed to becoming lame. In still further embodiments, an increased ratio of P2CP/CTX2 in the saliva sample indicates that the pig is lame or is predisposed to becoming lame. In still further embodiments, an increased ratio of P2CP/C2C in the saliva sample indicates that the pig is lame or is predisposed to becoming lame.
(c) Treating Lameness

In some embodiments, the methods further comprise administering at least one organic mineral to the pig that is lame or is predisposed to becoming lame, wherein administering the at least one organic mineral reduces symptoms of lameness as compared to administering at least one inorganic mineral to a comparable pig afflicted with lameness.

Organic minerals are detailed above in section (III)(a). In specific embodiments, the at least one organic mineral is a metal chelate of MHA, which are detailed above in section (III)(a).

Administration of the at least one organic mineral reduces symptoms or lameness and may change levels of biomarkers as described above in section (III)(b).

In some embodiments, administering the at least one organic mineral significantly increases the saliva level of OC, whereas administering at least one inorganic mineral to a comparable pig decreases or results in no significant change in the saliva level of OC. In other embodiments, administering the at least one organic mineral significantly increases the saliva level of P2CP, whereas administering at least one inorganic mineral to a comparable pig increases or results in no significant change in the saliva level of P2CP. In other embodiments, administering the at least one organic mineral significantly increases the ratio of P2CP/CTX2, whereas administering at least one inorganic mineral to a comparable pig increases or results in no significant change in the ratio of P2CP/CTX2.

### DEFINITIONS

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The term “about,” particularly in reference to a given quantity, is meant to encompass deviations of plus or minus five percent.

The terms “treating” or “treatment,” as used herein, refer to reducing, alleviating, ameliorating, or inhibiting the symptoms of lameness; slowing, inhibiting, or reversing the progression of lameness; and/or delaying or preventing the onset or development of lameness.

As used herein, the term “detecting” refers to means for identifying individuals afflicted with or prone to becoming lame, but which do not exhibit overt symptoms of lameness. Early detection may result in early treatment and/or lifestyle changes and/or may reduce the severity of lameness and/or may increase the quality of life of the individual.

### EXAMPLES

The following examples illustrate various embodiments of the present disclosure.

### Example 1: Quantification of Lameness with Serum Biomarkers

A database of gait scores and serum biomarkers was created based on a total of five trials including 525 pigs and 1320 observations. Gait score was measured on a 5-point scoring system ranging from 0 to 4 including half-scores (e.g., 0, 0.5, 1, 1.5, 2, 2.5, 3, 3.5, 4) to define the degree of lameness. For each pig, lameness was assessed at three times during the trial. Although the time of the measurements differed between each trial, each trial lasted approximately two months (e.g., 54-70 d). Mean levels of each biomarker were determined every time the gait score of the pig was measured. The biomarker levels were then normalized (mean=0, standard deviation=1) using a SAS program. Pearson correlation coefficients were used to determine which biomarkers significantly correlated with gait score.

FIG. 1 shows the correlation of statistically significant biomarkers with gait score. The x-axis shows the gait score, and the y-axis shows the standard deviation of the biomarker from the mean; i.e., if the y-value of a biomarker is positive, then the level of the biomarker is higher than the mean, whereas if the y-value of a biomarker is negative, then the level of the biomarker is lower than the mean. The significant biomarkers include C2C, P2CP, CTX1, OC the ratio of P2CP to CTX2, and the ratio of P2CP to C2C. The data show that above a gait score of about 2, the standard deviation of the biomarkers increases to above ±0.5.

It can be seen from FIG. 1 that the level of bone synthesis (e.g., OC) and cartilage degradative biomarkers (e.g., C2C, CTX1) decreased as gait score increased, the level of cartilage synthesis biomarkers (e.g., P2CP) increased as gait score increased, and the ratio of cartilage synthesis biomarkers to cartilage degradative biomarkers (e.g., P2CP/CTX2, P2CP/C2C) increased as gait score increased.

### Example 2: Treating Lameness Induced by Fast Growth Rate

A study was conducted in 144 healthy barrows, in which serum biomarkers were measured over 69 days at day 0, day 28, and day 69. Fast growth was induced in some pigs as explained below.

The study was a 2×2 factorial study. The first factor was diet. Pigs were fed either inorganic trace minerals (ITM) or Mintrex° (MTX), an organic mineral. The ITM included a ratio of 120-20-40 ppm Zn—Cu—Mn. The MTX included a ratio of 80-10-20 ppm Zn—Cu—Mn.

The second factor was feeding ractopamine (RAC; a beta-adrenergic agonist) to induce fast growth. Pigs were fed ractopamine at either 0 ppm or 10 ppm during the last 25 days of the trial.

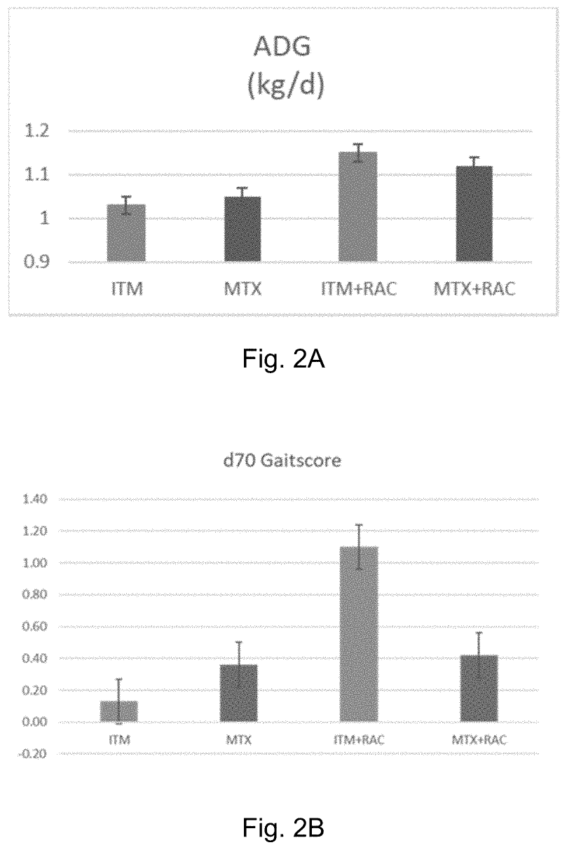

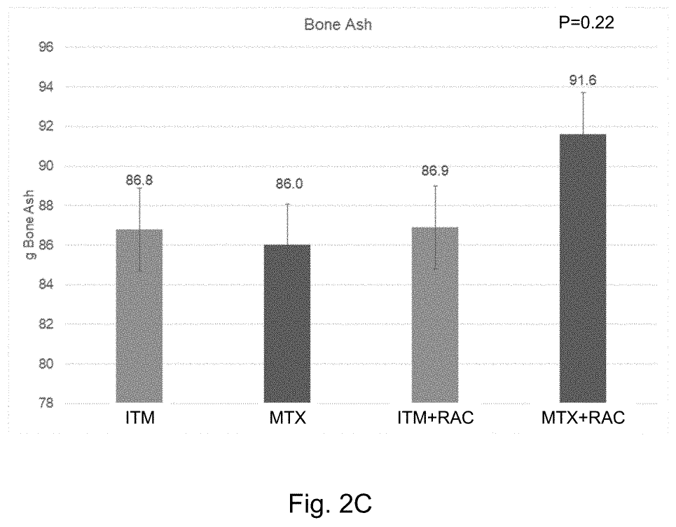

FIG. 2A shows the average daily growth (ADG) of the pigs in the study and FIG. 2B presents the gait score on Day 70. FIG. 2C shows the amount of bone ash in the pigs at the end of the study.

As expected, ADG increased for pigs fed RAC regardless of diet; however, gait score increased substantially for pigs fed ITM and RAC as compared to pigs fed MTX and RAC. In fact, pigs fed MTX and RAC did not show an elevated gait score as compared to pigs fed MTX alone, indicating that MTX maintained a normal healthy gait without compromising growth rate in response to RAC. Rather, MTX reduced the incidence and severity of lameness in pigs with fast growth. Pigs fed RAC were expected to have higher bone ash to support increased ADG, increased body weight, and increased lean deposition compared to pigs fed diets without RAC. However, pigs fed ITM and RAC had similar bone ash as pigs not fed RAC. Pigs fed MTX and RAC had 5% higher bone ash vs pigs fed ITM and RAC.

Because the biomarker data were not normally distributed, the individual markers were logarithmically transformed for evaluation in the model. In addition, because there were day 0 differences for some of the biomarkers, day 0 biomarker concentrations were used as a covariate. Next, main effects and interactions were determined using a mixed model analysis of variance (ANOVA). Table 1 means presented are the inverse of the log-transformed values. The biomarkers Table 1 include osteocalcin, CTX1, the ratio of osteocalcin to CTX1, C2C, CTX2, P2CP, the ratio of P2CP to C2C, and the ratio of P2CP to CTX2.

TABLE 1
Serum Biomarker Treatment Means as Affected by Treatment and Day

| RAC | | CTX1 | OC | OC/ | C2C | CTX2 | P2CP | P2CP/ | P2CP/ |
|---|---|---|---|---|---|---|---|---|---|
| (ppm) | MIN | (ng/mL) | (ng/mL) | CTX1 | (ng/mL) | (ng/mL) | (ng/mL) | C2C | CTX2 |
| 0 | ITM | 158.5 | 75.5 | 0.46 | 9.0 | 0.72^{a} | 1.86^{ab} | 0.23^{ab} | 2.51 |
| 0 | MTX | 158.5 | 76.4 | 0.49 | 8.8 | 0.71^{a} | 2.09^{a} | 0.23^{ab} | 2.82 |
| 10 | ITM | 158.5 | 79.6 | 0.50 | 8.2 | 0.65^{by} | 1.74^{b} | 0.20^{b} | 2.63 |
| 10 | MTX | 154.9 | 76.3 | 0.49 | 8.3 | 0.69^{abx} | 1.66^{b} | 0.28^{a} | 2.57 |

P-values

| | | | | | | | | |
|---|---|---|---|---|---|---|---|---|
| Covar | 0.9695 | <0.0001 | <0.0001 | 0.1965 | 0.6847 | <0.0001 | 0.8454 | 0.0028 |
| Trt | 0.4707 | 0.5043 | 0.2827 | 0.4726 | 0.0002 | 0.0119 | 0.0437 | 0.4758 |
| Day | <0.0001 | 0.0004 | 0.0840 | 0.0021 | 0.0531 | <0.0001 | <0.0001 | <0.0001 |
| Trt*day | 0.1688 | 0.8058 | 0.7829 | 0.0486 | <0.0001 | 0.9071 | 0.2326 | 0.1412 |

*Values with different superscripts (^{a}, ^{b}) are significantly different (P < 0.05); trend (^{x}, ^{y}; P < 0.10).

As discussed in Example 1, increased gait score was significantly correlated with increasing serum concentrations of P2CP and increasing ratio of serum concentrations P2CP to C2C. Although lameness was increased in pigs fed RAC and ITM, as shown in FIG. 2B, serum concentrations of P2CP were not higher or not different in pigs fed RAC and ITM vs other treatments. Likewise, the ratio of serum concentrations of P2CP to C2C was not highest for pigs fed ITM and RAC, so the direction of this cartilage ratio in Table 1 did not agree with the increase in this ratio shown in FIG. 1. The increase or decrease of the biomarkers from the mean with increasing lameness may vary from trial to trial, but in general dietary intervention should result in an increase or decrease that is opposite of the change observed with increasing lameness in the absence of dietary intervention. However, all findings for Mintrex in this trial (e.g., reduction in gait score without slowing growth rate, increased bone ash, etc.) outweighed the ITM findings (e.g., negative direction observed for P2CP/C2C in pigs fed RAC and Mintrex vs RAC and ITM) with respect to lameness.

### Example 3: Saliva Biomarkers

A trial was conducted to evaluate biomarkers in saliva. The trial included 36 nursery pigs who were fed a P-deficient diet (0.18% P reduction compared to adequate control). At the end of the trial, 18 of the 36 pigs were lame, the remaining 18 pigs were not lame. Lameness was evaluated based on gait score ranging from 0 to 4 (0=healthy, 4=severely lame).

Saliva and serum samples were collected on the last day of the trial. Saliva was collected by offering the pigs a sponge on the end of a stick. Pigs were allowed to chew the sponge for 1-2 minutes.

The saliva samples were analyzed to determine the concentration of biomarkers in the samples. Results for saliva biomarkers are shown in Table 2.

TABLE 2
Saliva Biomarkers

| | Gait | CTX1 | Osteocalcin | C2C | P2CP | | |
|---|---|---|---|---|---|---|---|
| | Score | (ng/mL) | (ng/mL) | (ng/mL) | (ng/mL) | OC/CTX1 | P2CP/C2C |
| Lame | 3.61 | 44.8^{x} | 30.6 | 8.71 | 3.26 | 1.19 | 0.365 |
| Sound | 1.67 | 25.5^{y} | 32.6 | 7.83 | 1.98 | 1.41 | 0.275 |
| SEM | .13 | 7.8 | 5.37 | 1.14 | 0.8 | 0.3 | 0.08 |
| P-value | <0.0001 | 0.0998 | 0.7911 | 0.5937 | 0.2796 | 0.6223 | 0.4400 |

*Values with different superscripts (a, b) are significantly different (P < 0.05); trend (^{x}, ^{y}; P < 0.10).

The serum samples were analyzed to determine the concentration of biomarkers in the samples. Results for serum biomarkers are shown in Table 3.

TABLE 3
Serum Biomarkers

| | Gait | CTX1 | Osteocalcin | C2C | P2CP | | |
|---|---|---|---|---|---|---|---|
| | Score | (ng/mL) | (ng/mL) | (ng/mL) | (ng/mL) | OC/CTX1 | P2CP/C2C |
| Lame | 3.61 | 187.4 | 67.3 | 22.1^{a} | 0.63 | .352 | 0.029^{y} |
| Sound | 1.67 | 196.5 | 69.8 | 18.9^{b} | 0.67 | .363 | 0.036^{x} |
| SEM | .13 | 10.8 | 6.4 | 1.02 | 0.05 | 0.03 | 0.003 |
| P-value | <0.0001 | 0.5571 | 0.7907 | 0.0442 | 0.6155 | 0.8066 | 0.0968 |

*Values with different superscripts (^{a}, ^{b}) are significantly different (P < 0.05); trend (^{x}, ^{y}; P < 0.10).

As shown in Table 2 and Table 3 lame pigs had a mean gait score 2-fold higher than sound (non-lame) pigs. Significant differences in saliva biomarkers between sound pigs and lame pigs were observed for CTX1, which was increased in lame pigs. This increase in CTX1 with phosphorus deficiency observed in saliva is in agreement with Sorensen et al. (2018) who also reported increased CTX1 in serum in pigs fed P-deficient diets. In contrast, no significant differences in CTX1 were observed in serum samples. However, significant differences in serum concentration of C2C and the ratio of serum concentration of P2CP and C2C were observed between sound and lame pigs.

It is unclear from these results why there are differences in the increase or decrease of biomarkers when measured in different biological fluids. This may be the result of timing (i.e., changes occurring at different rates in different biological tissues), the predicted cyclical nature of the biomarker time course, or another unaccounted-for variable.

### Example 4: Treating Lameness Induced by Fast Growth Rate

A trial was conducted to measure outcomes of inorganic trace minerals (ITM), organic trace minerals (OTM) and Mintrex (MTX) diets. A total of 144 barrows (average initial body weight approx. 50 kg) were randomized to treatment based on body weight. The study design consisted of 4 treatments: 1) inorganic trace minerals (ITM), sulfates, 2) organic trace minerals (OTM) Competitor #1, 3) OTM Competitor #2, and 4) Mintrex. OTM Competitor #1 included a non-specific amino acid complex. OTM Competitor #2 included a glycinate (i.e., a complex including a glycine ligand). Ractopamine (RAC) was fed in an amount of 10 mg/kg the last 28 d of study for all four dietary treatments to induce fast growth. All OTMs provided 80-10-20 ppm of Zn—Cu—Mn, respectively, whereas ITM was provided at 120-20-40 mg/kg diet for a total of 4 treatments which were fed for 70 days. Measurements included growth performance and gait scoring (0-4; GS defined as lame) measured at baseline, day 7, day 36 and day 70.

Pigs were fed a 3-phase diet regimen during the study and formulated to meet PIC requirements for developing gilts. Diets containing ractopamine contained higher concentrations of amino acids and protein per Paylean label guidelines. The primary dietary difference was trace mineral source. Methionine hydroxy analog (MHA) was added to competitor treatments to equalize methionine concentrations. Description of experimental treatments in phases 1-3 are displayed in Table 4. During the last month of the study, when pigs were approximately 100 kg, all pigs were switched to diets containing ractopamine (RAC; 10 mg/kg diet). All diets met

NRC requirements for growing/finishing pigs. Timing of diet switch and key measurements is shown in Table 5.

TABLE 4
Description of Dietary Treatments

| | | RAC (mg/kg); |
|---|---|---|
| | | fed last 4 wk |
| Treatment | Mineral source, Zn—Cu—Mn | of trial |
| 1 | ITM (120-20-40) | 10 |
| 2 | OTM Competitor #1 Zn/Cu/Mn (80-10-20) | 10 |
| 3 | OTM Competitor #2 Zn/Cu/Mn (80-10-20) | 10 |
| 4 | MTX (80-10-20) | 10 |

TABLE 5
Timing of diet switch and key measurements

| | Approx. | | | Gait | Serum & saliva |
|---|---|---|---|---|---|
| Day | BW | Performance | Diet switch | score | biomarkers |
| 0 | 54 | X | | X | X |
| 7 | 64 | | | X | X |
| 14 | 74 | X | Phase 2 | | |
| 28 | 91 | X | | | |
| 36 | 97 | | | X | X |
| 42 | 104 | X | Phase 3 (all 4 | | |
| | | | trts + RAC) | | |
| 56 | 118 | X | | | |
| 70 | 133 | X | EOS-collect right | X | X |
| | | | front metacarpal | | |
| | | | post-mortem* | | |

*Front feet were lost in shipping, so no DEXA/bone ash results were obtained in this trial.

Measurements. Pigs were fed dietary treatments for 70 days. Animal performance was measured every 2 weeks of the trial. Lameness measurements (gait score, serum and saliva) were collected at baseline, day 7, day 36, and day 70. Gait score was evaluated using the 5 pt scoring system in Table 7.

TABLE 6
5-point Scoring System Used for Subjective
Evaluation of Gait in Gilts/Sows

| Lameness | |
|---|---|
| score | Description |
| 0 | Gilt moves freely and uses all 4 limbs and feet evenly |
| 1 | Gilt shows weight-shifting activities away from affected |
| | limb upon standing but shows no lameness or limping when |
| | walking; abnormal stride length; movement no longer |
| | fluent; gait may or may not be slightly slowed |
| 2 | Gilt obviously shifts weight away from affected limb when |
| | standing & shows limping or adaptive behavior when walking |
| | (head bob, quickened step on affected limb, arched back); |
| | gait is slowed & asymmetrical; 1 side swings more than |
| | other side |
| 3 | Gilt is reluctant to stand and/or walk, obvious limp |
| | & adaptive behaviors when walking; shortened stride, |
| | visibly carrying limb; caudal swagger is observed |
| 4 | Gilt is non-weight bearing on the affected limb when |
| | either standing or walking |

Karriker et al. 2013

Serum collections. Approximately 2 tubes (20 mL) of blood from each pig were collected on days 0, 7, 35 and 70 using red top (clotted) tubes and centrifuged for 15 minutes at 3000 rpm at room temperature. Two aliquots (1.5-1.8 mL per aliquot) for biomarkers of cartilage and bone metabolism were collected (P2CP, CTX2, osteocalcin, C2C, CTX1) and assayed.

Saliva collection. Saliva was collected using a sponge (1 cm×1 cm in size) from 18 pigs per treatment (trt) (total =72). The sponge was clipped to a metal rod and pigs were allowed to chew on sponge for 1-2 min. The sponge from each pig was placed in salivette tubes. Saliva tubes were centrifuged for 10 min at 3000×g. The supernatant was collected in Eppendorf tubes of 1.5 mL and the sediment discarded.

### Example 5: Lameness Incidence between Pigs Fed Mintrex and other OTMs

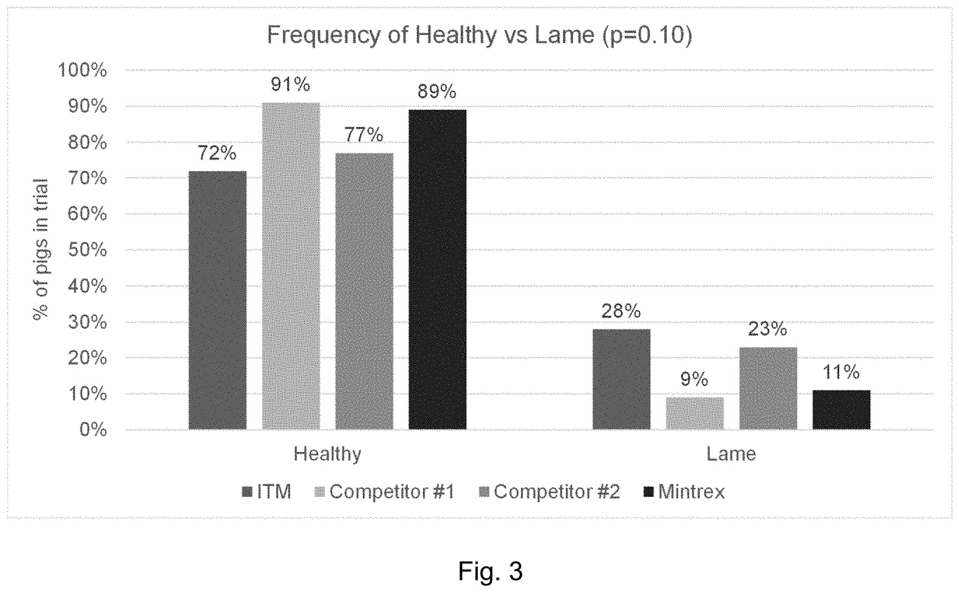

FIG. 3 depicts how lameness incidence on day 70 was lower for Mintrex and OTM Competitor #1 compared to ITM and OTM Competitor #2 (P=0.10). Overall lameness percentage in this trial (GS≥2) was 18%. Average gait score on day 70 did not differ between treatments (P=0.51), but bodyweights (day 70) did (see Table 7). Pigs fed OTM Competitor #2 and OTM Competitor #1 were highest, Mintrex was intermediate (significantly lower than OTM Competitor #2, but not different from OTM Competitor #1 or ITM). Pigs fed ITM had lower body weight than pigs fed OTM Competitor #1 or OTM Competitor #2.

TABLE 7
End Bodyweight and Gait Score

| | | Body Weight day 70 | Gait Score |
|---|---|---|---|
| | Treatment | (kg) | Day 70 |
| 1 | ITM | 126.1^{c} | 0.69 |
| 2 | OTM | 132.3^{ab} | 0.46 |
| | Competitor #1 | | |
| 3 | OTM | 133.8^{a} | 0.63 |
| | Competitor #2 | | |
| 4 | Mintrex | 129.1^{bc} | 0.39 |
| | SEM | 1.6 | 0.16 |
| | P-value | 0.0033 | 0.5057 |

*Values with different superscripts (^{a}, ^{b}, ^{c}) are significantly different (P < 0.05).

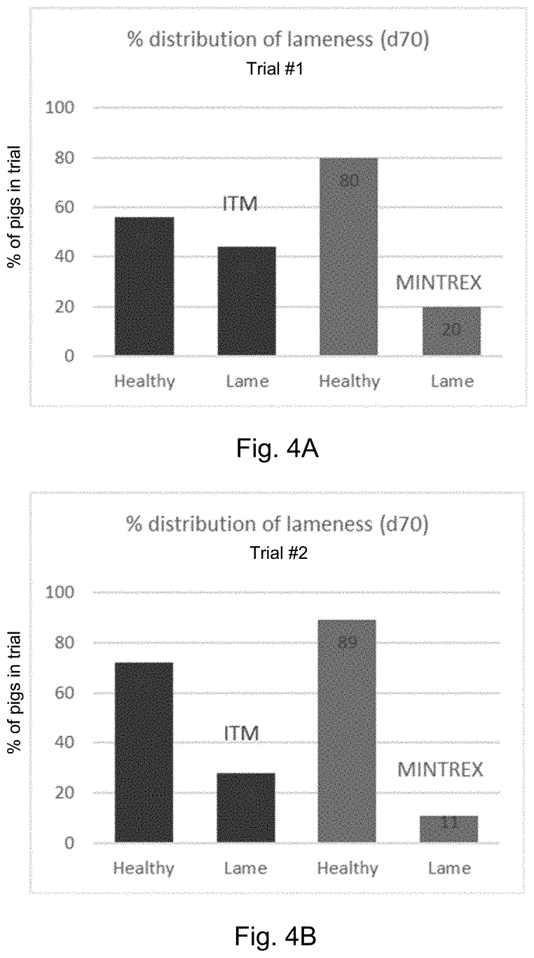

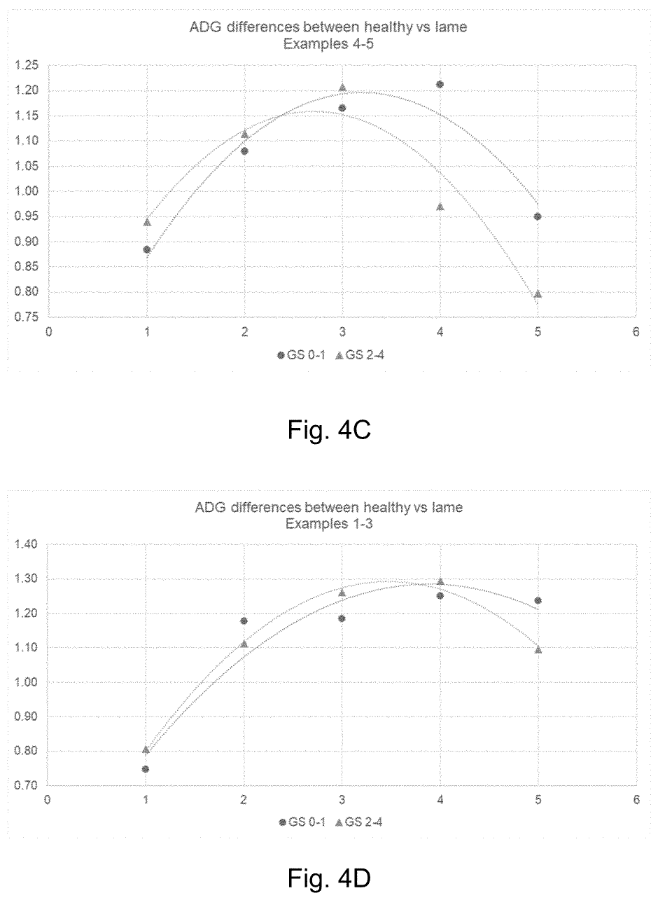

It was further observed that average daily growth (ADG) and gait score on day 70 was higher in the study described in Example 2 (see Table 8). Faster growth is associated with higher lameness incidence, so it was not surprising that lameness (defined as GS≥2) was lower in the trial described in Example 4 compared to that described in Example 2. ITM+RAC and Mintrex+RAC were common to both trials (both bolded in Table 8). Despite differences in lameness incidence between the two trials, it is noteworthy that the percent reduction in lameness due to Mintrex between trials (see FIGS. 4A and 4B; 55% and 61%, respectively) was similar.

FIGS. 4C and 4D show the difference in average daily growth between healthy and lame (GS≥2) in both RAC trials based on final gait score. Both trials were of similar duration (70 days) and pigs had similar initial body weight. Faster growth rates, higher gait scores, and higher percentage of lameness was observed in the trial described in Example 2 vs the trial described in Example 4, but comparing ADG between healthy vs lame pigs, differences were minimal in the trial described in Example 2. It was only the last two weeks of the trial that the ADG declined for lame pigs, but ADG was maintained in healthy pigs. ADG over entire study duration (ADGdO-70) was not different between healthy vs lame pigs (e.g., ADG=1.13 g/day).

In Example 4, marked differences in ADG between healthy vs lame were observed the last 4 weeks of the trial (days 45-70; only 40 g difference overall ADG (d0-70)). Regardless of whether the pigs were healthy or lame, the ADG declined for both populations during the last 2 weeks of the trial for healthy pigs and the last 4 weeks of the trial for lame pigs. This pattern differed from Example 2. Interestingly, in Example 4, there was no significant relationship between gait score and serum biomarkers (for entire trial dataset), which may have been due to lower ADG and lower lameness incidence. The percentage of lame pigs at day 70 (GS≥2) was, however, similar between the two trials.

ADG did differ between treatments (see Table 8). Specifically, higher growth performance was achieved for pigs fed OTM Competitor #1 and OTM Competitor #2, Mintrex was intermediate and lowest for ITM. Based on overall ADG (d0-70), Mintrex was not different from other OTMs or ITM. Based on body weights on day 70, pigs fed Mintrex had significantly lower body weight vs OTM Competitor #2, but was not different from OTM Competitor #1 or ITM.

TABLE 8
Comparison of Growth Rates, Gait Scores, and Incidence
of Lameness between Two RAC lameness trials

| Example 4 | | | | Example 2 | | | | | |
|---|---|---|---|---|---|---|---|---|---|
| | ADG | | % | | | | ADG | | |
| | (g/day; | GS | lame | | RAC | Trace | (9/day; | GS | % lame |
| Treatment | d 0-d 70) | (d 70) | (d 70) | Trt | (ppm) | min | d 70) | (d 70) | (d 70) |
| ITM | 1.00^{b} | 0.69 | 28^{a} | 1 | 0 | ITM | 1.03^{b} | 0.13^{b} | 3^{b} |
| OTM | 1.09^{a} | 0.46 | 9^{b} | 2 | 0 | Mintrex | 1.05^{b} | 0.36^{b} | 14^{b} |
| Competitor | | | | | | | | | |
| #1 | | | | | | | | | |
| OTM | 1.11^{a} | 0.63 | 23^{a} | 3 | 10 | ITM | 1.15^{a} | 1.10^{a} | 44^{a} |
| Competitor | | | | | | | | | |
| #2 | | | | | | | | | |
| Mintrex | 1.04^{ab} | 0.39 | 11^{b} | 4 | 10 | Mintrex | 1.12^{a} | 0.42^{b} | 20^{b} |

*Values with different superscripts (^{a}, ^{b}) are significantly different (P < 0.05).

### Example 6: Mintrex vs Competitor OTMs (Serum and Saliva Biomarker Levels)

Biomarker findings from serum (see Table 9) showed significant treatment effects for OC (P=0.079) and P2CP (P=0.034). Biomarker findings from saliva showed non-significant treatment effects for osteocalcin, but there was a significant effect of treatment for P2CP (see Table 12; P=0.006) which aligned with findings from saliva. In general, there was good agreement between serum and saliva with respect to cartilage biomarkers. In addition, in saliva, significant treatment effects were also observed for P2CP/C2C (P=0.0019) and P2CP/CTX2 (P=0.0003).

TABLE 9
Significant differences between Mintrex and
other treatments for serum OC and P2CP

| | OC | P2CP | | | CTX1 |
|---|---|---|---|---|---|
| | (ng/mL) | (ng/mL) | P2CP/C2C | P2CP/CTX2 | (ng/mL) |
| ITM | 64.6^{ab} | 0.99^{b} | 0.069 | 0.955 | 125.9 |
| OTM | 58.9^{b} | 1.12^{ab} | 0.078 | 1.071 | 123.0 |
| Competitor #1 | | | | | |
| OTM | 64.6^{ab} | 1.05^{ab} | 0.071 | 0.912 | 125.9 |
| Competitor #2 | | | | | |
| Mintrex | 69.2^{a} | 1.27^{a} | 0.085 | 1.175 | 125.9 |
| P-value (Trt) | 0.0793 | 0.034 | 0.2537 | 0.0966 | 0.654 |

*Values with different superscripts (^{a}, ^{b}) are significantly different (P < 0.05).

TABLE 10
Significant differences between Mintrex and other
treatments for saliva cartilage synthesis and
S:D ratios (P2CP and P2CP/C2C, P2CP/CTX2)

| | OC | P2CP | | | CTX1 |
|---|---|---|---|---|---|
| Trt | (ng/mL) | (ng/mL) | P2CP/C2C | P2CP/CTX2 | (ng/mL) |
| ITM | 7.4 | 2.43^{b} | 0.23^{b} | 0.66^{b} | 84.5 |
| OTM | 7.7 | 2.21^{b} | 0.19^{b} | 0.49^{b} | 85.8 |
| Competitor #1 | | | | | |
| OTM | 7.5 | 2.53^{b} | 0.21^{b} | 0.62^{b} | 88.4 |
| Competitor #2 | | | | | |
| Mintrex | 7.6 | 3.98^{a} | 0.32^{a} | 0.95^{a} | 92.8 |
| P-value (Trt) | 0.9608 | 0.006 | 0.0019 | 0.0003 | 0.9818 |

*Values with different superscripts (^{a}, ^{b}) are significantly different (P < 0.05).

It was noted that saliva and serum biomarkers did not completely align with bone biomarkers in this study. Specifically, the osteocalcin (OC) concentrations in saliva (see Table 10) were approximately 10-fold lower than serum (see Table 9). Not only were concentrations different, but the direction/ranking between treatments also differed between biological fluids (e.g., OTM Competitor #1 was lowest in serum, but highest in saliva for OC). There were no significant treatment effects for saliva bone synthesis biomarker (OC; Table 10), which disagreed from serum bone biomarker findings. The lower incidence of lameness in this trial may explain the lack of treatment differences observed in saliva for OC. Based on the results in the trial described in Example 4, comparing serum and saliva biomarkers, saliva cartilage biomarkers agreed closely with serum and may be more sensitive (lower p-values and higher concentrations for saliva P2CP, P2CP/C2C and P2CP/CTX2.

In contrast to Example 3 (saliva from P-deficient pigs), no treatment differences were observed between treatments in CTX1 in saliva or serum in the trial described in Example 4. In addition, lameness severity (gait score) was lower compared to the trial described in Example 3. One main difference between trials is that phosphorus deficiency is known to cause bone degradation/loss (e.g., elevated CTX1); whereas, in Example 4, diets were nutritionally adequate.

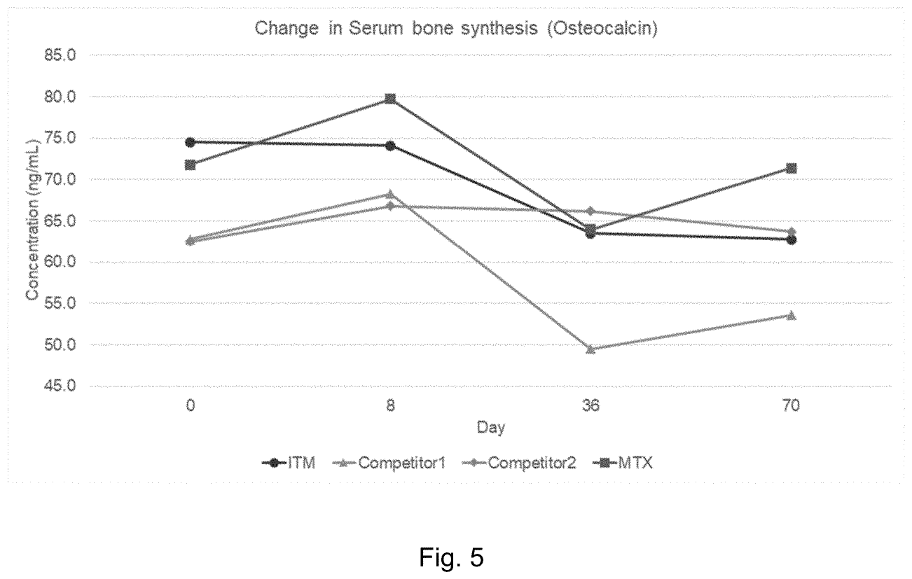

FIG. 5 shows the change in OC in serum for all four treatments over the duration of the study. Mean concentrations (ng/mL) for OC were highest for Mintrex which differed from OTM Competitor #1 (P<0.05). The other treatments were intermediate and not different from OTM Competitor #1 or Mintrex. Statistics showed treatment differences for OC at day 0. For this reason, all biomarkers were re-analyzed using day 0 biomarker concentrations as a covariate to correct for day 0 treatment differences. The adjusted/corrected means are shown in Tables 9 and 10.

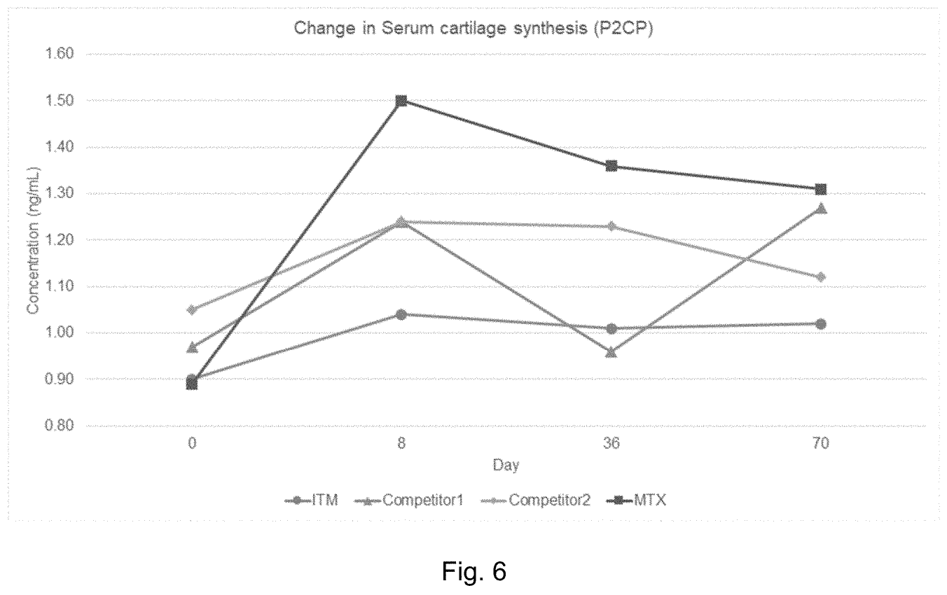

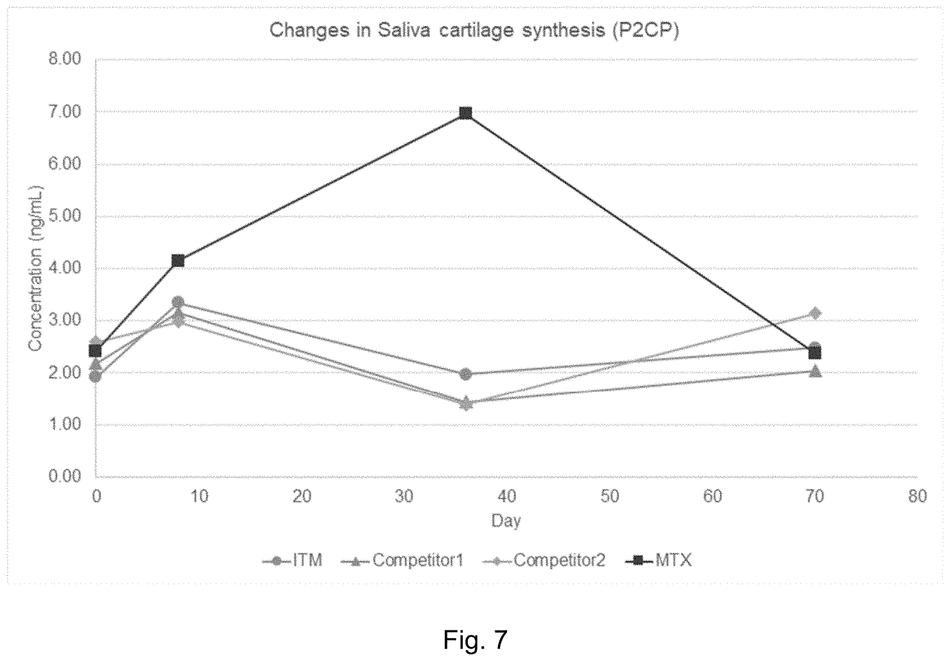

FIG. 6 shows the change in P2CP that occurred during the trial described in Example 4 between all four treatments in serum. Although the pattern differed (peak concentrations occurred at different timepoints between saliva (see FIG. 7) and serum), overall findings for both serum and saliva show non-significant differences in average treatment means for P2CP between ITM, OTM Competitor #1 and OTM Competitor #2. Based on saliva findings, pigs fed Mintrex had significantly higher P2CP than all other treatments (see Table 10). Based on serum, Mintrex is only significantly different from ITM, with the other treatments being intermediate and not different from each other. The rankings of the four treatments align well between saliva and serum for cartilage biomarkers.

### Example 7: Relationship between Biomarkers and Gait Score in Population with High Lameness Incidence due to Bacterial Infection

Another trial was performed wherein grower/finisher pigs were fed incremental levels of Mintrex Zn (0, 50, 75 and 100 ppm) to determine if Zn had beneficial effects on gait score and biomarkers and, if yes, to determine an optimal Zn dose for grower/finisher pigs. However, this trial was terminated early because of animal welfare concerns. Approximately 2 weeks following start of trial, pigs started showing signs of lameness. Pigs were treated with Banamine and LA300, but never fully recovered. The trial was aborted on day 40 (4 weeks earlier than originally planned).

TABLE 11
Gait score distribution by timepoint.

| | | | GS distribution (# of pigs) | | | | | # pigs analyzed for serum |
|---|---|---|---|---|---|---|---|---|
| Date | Timepoint | (n) | 0 | 1 | 2 | 3 | 4 | BM's |
| 2-22 | Day 0 | 140 | 140 | 0 | 0 | 0 | 0 | 51 |
| 3-2 | Day 7 | 140 | 51 | 61 | 24 | 4 | 0 | 58 |
| 3-23 | Day 28 | 35 | 7 | 12 | 11 | 7 | 2 | 35 |
| 4-4 | Day 40 | 66 | 0 | 20 | 26 | 20 | 0 | 66 |

Originally, this trial started with 140 pigs, however, because of concerns that pigs might have a contagious condition, the personnel dealing with the pigs and conducting the trial was reduced to two and the focus of the personnel was to treat pigs and less time was available for data collection. Only a subsample of pigs were scored for gait score and blood samples from day 28 onward (see Table 11). A number of diagnostic tests by veterinarians were conducted to identify cause of lameness. Seneca virus, porcine circovirus, swine influenza, mycoplasma virus, foot and mouth (FMD), African swine fever (ASF) were all ruled out. It was suspected by the veterinarian that the morbidity and lameness was attributed to Strep suis. Note that the percentage of lame pigs (GS≥2) was 20% as early as day 7 and 70% by end of trial. Thus, although not the original intent of this trial, the conditions simulated a disease outbreak that caused lameness, and thus simulated conditions that sometimes occurs naturally. This type of lameness (infectious and inflammatory) may result in a more chronic lameness condition compared to lameness induced by fast growth.

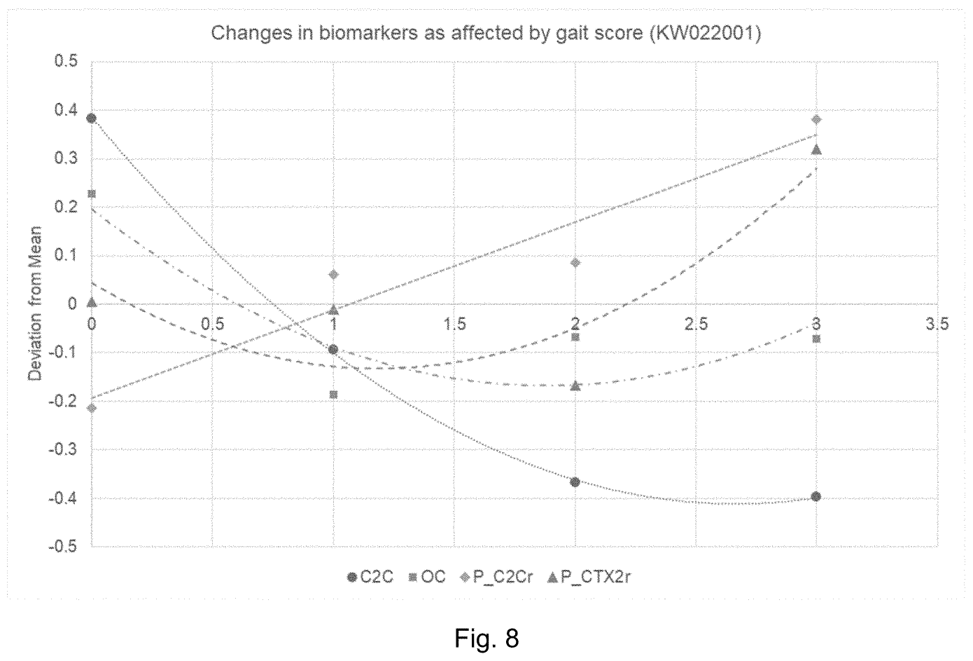

FIG. 8 shows a significant relationship between biomarkers and lameness similar to what had been observed in prior trials. The only difference between findings from this and previous trials was that the biomarker OC was significant, whereas CTX1 was not. There also appeared to be more variability at lower gait scores and differences observed between GS0 and GS2 (C2C for example) observed in this study compared to prior trials. Table 12 below shows which biomarkers had a significant relationship to gait score based on Pearson correlation coefficients, as well as p-values from analysis of variance (ANOVA) demonstrating a significant effect of gait score. Lastly, the magnitude of change between GS 3 and GS 0 is also shown.

TABLE 12
Pearson correlation coefficients showing relationship between GS and biomarkers

| Pearson correlation | Biomarkers with significant correlation to gait score | | | | | | |
|---|---|---|---|---|---|---|---|
| analysis | OC | CTX1 | CTX2 | C2C | P2CP/C2C | P2CP/CTX2 | OC/CTX1 |
| Correlation coefficient (r) | −0.209 | −0.098 | −0.133 | −0.334 | 0.23 | 0.122 | −0.153 |
| P-value | 0.002 | 0.1505 | 0.052 | <0.0001 | 0.0007 | 0.074 | 0.025 |
| (n) | 215 | 215 | 215 | 216 | 214 | 214 | 214 |

TABLE 13
ANOVA Showing Significant Relationship between GS and Biomarkers

| | OC | CTX1 | CTX2 | C2C | P2CP/C2C | P2CP/CTX2 | OC/CTX1 |
|---|---|---|---|---|---|---|---|
| Magnitude of change between | 17%↓ | 8%↓ | 11%↓ | 20%↓ | 50%↑ | 30%↑ | 13%↓ |
| healthy & lame | | | | | | | |
| P-value | 0.0163 | 0.0218 | 0.2071 | <0.0001 | 0.0034 | 0.0423 | 0.1848 |

### Example 8: Summary of Data from Examples 1-7

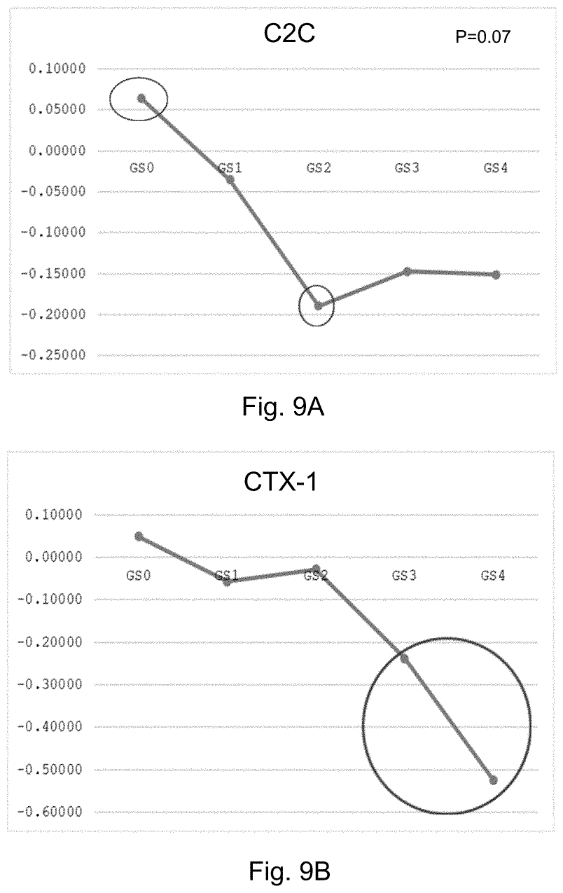

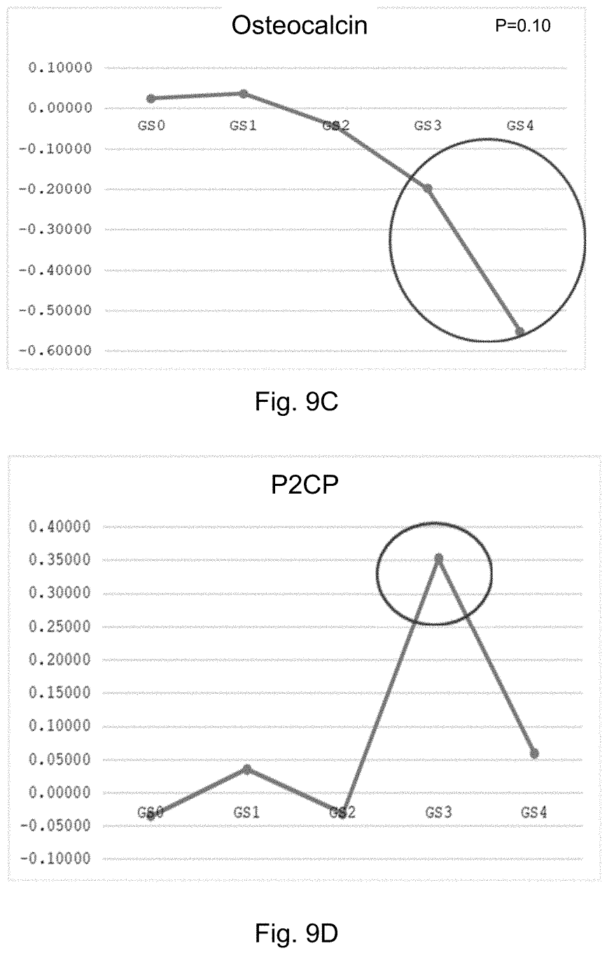

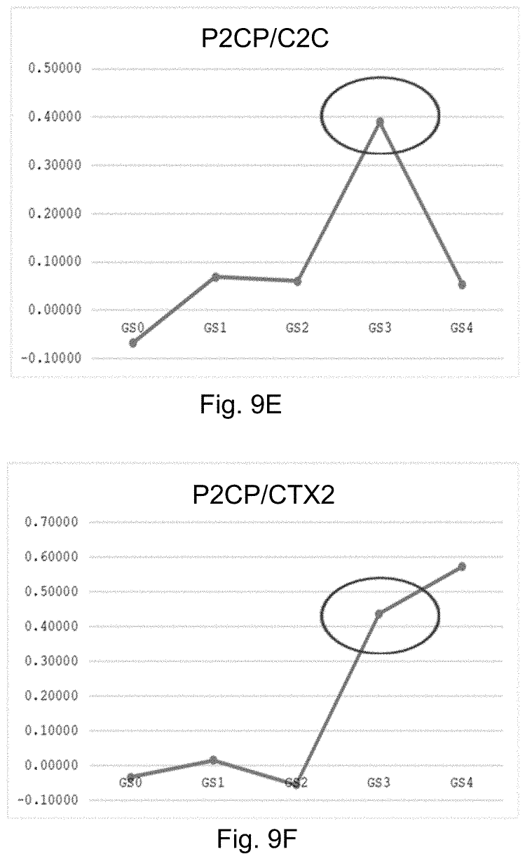

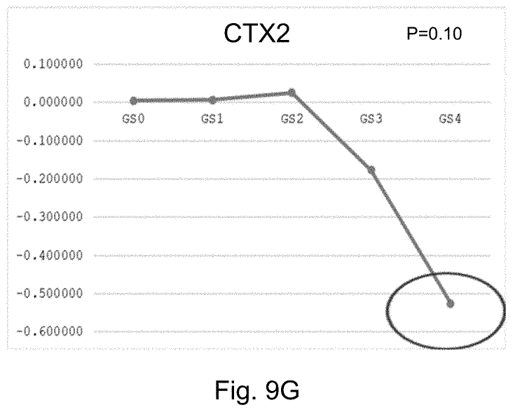

Looking at the relationship between biomarkers and gait score, our previous conclusions were that biomarkers were similar between GS 0-GS 2 and changes generally did not occur until GS was above 2. Data summarized in FIG. 1 and then broken down in FIGS. 9A-9G further suggest that the biggest changes in most biomarkers occur around GS 3 (e.g., P2CP, P2CP/C2C, P2CP/CTX2, OC and CTX1). However, in the case of C2C, there was a trend for GS 0 to be higher than 0 and different from other GS's; additionally, around GS 2, there was a marked reduction in C2C concentrations. C2C may be one of the most sensitive markers for early detection of lameness with changes occurring at lower gait scores. Across multiple trials, this marker has consistently been shown to differ between healthy vs lame pigs. However, the number of GS 4s in the dataset is limited which may affect the variability and accuracy of data past GS 3.

Having described the invention in detail, it will be apparent that modifications and variations are possible without departing from the scope of the invention defined in the appended claims.

What is claimed is:

1. A method for quantifying lameness in a pig, the method comprising:
(a) determining a level of at least one biomarker and/or a ratio of two biomarkers in a serum sample from the pig, wherein the at least one biomarker is C2C, CTX1, CTX2, OC, or P2CP, and the ratio of two biomarkers is OC/CTX1, P2CP/C2C, or P2CP/CTX2;
(b) generating a test score by standardizing the data from (a) to have a mean of 0 and a standard deviation of 1; and
(c) using the test score to quantify lameness, wherein increasing deviation of the test score from the mean correlates with increased lameness.

2. The method of claim 1, wherein an increase or decrease from about 0.1 to about 0.2 units from the mean correlates with a lameness gait score of about 2.0.

3. The method of claim 1, wherein an increase or decrease of about 0.3 to about 0.5 units from the mean correlates with a lameness gait score of about 3.0.

4. The method of claim 1, wherein an increase or decrease of about 0.5 units from the mean or greater correlates with a lameness gait score of about 4.0.

5. The method of claim 1, wherein the relationship between test scores and gait scores is shown in FIG. 1.

6. The method of claim 1, wherein increasingly positive test scores for P2CP, P2CP/C2C, and P2CP/CTX2 correlate with increased lameness.

7. The method of claim 1, wherein increasingly negative test scores for C2C, CTX1, and OC correlate with increased lameness.

8. The method of claim 1, wherein the lameness is due to natural causes or due to fast body weight gain.

9. A method for treating lameness due to fast body weight gain in a pig, the method comprising administering at least one metal chelate of methionine hydroxy analog (MHA) to the pig, wherein administering the at least one metal chelate of MHA reduces symptoms of lameness as compared to administering at least one inorganic mineral to a comparable pig afflicted with lameness.

10. A method for treating lameness due to fast body weight gain in a pig, the method comprising administering at least one organic mineral to the pig, wherein administering the at least one organic mineral increases the serum level of OC, decreases the serum level of P2CP, decreases the serum ratio of P2CP/C2C, decreases the serum ratio of P2CP/CTX2, and/or increases the serum ratio of OC/CTX1 and reduces symptoms of lameness as compared to administering at least one inorganic mineral to a comparable pig afflicted with lameness.

11. The method of claim 10, wherein the at least one organic mineral comprises at least one metal ion bonded to at least one organic ligand to form a chelate, a complex, or a salt.

12. The method of claim 11, wherein the at least one metal ion is calcium, chromium, cobalt, copper, germanium, iron, lithium, magnesium, manganese, molybdenum, nickel, potassium, rubidium, sodium, tin, vanadium, zinc, or a combination thereof.

13. The method of claim 11, wherein the at least one organic ligand is an amino acid, an amino acid analog, a peptide, a protein, a protein hydrolysate, a hydroxy acid, an organic acid, a sugar alcohol, a polysaccharide, or a polynucleic acid.

14. The method of claim 10, wherein the at least one organic mineral is a metal amino acid complex, a metal proteinate, or a metal chelate of MHA.

15. The method of claim 12, wherein the at least one metal ion of the at least one organic mineral is copper, manganese, zinc, or a combination thereof.

16. The method of claim 10, wherein fast body weight gain is due to an increased level of at least one nutrient in the diet of the pig.

17. The method of claim 10, wherein fast body weight gain is due to addition of at least one growth promoter in the diet of the pig.

18. The method of claim 17, wherein the growth promoter is a beta-adrenergic agonist, an antimicrobial growth promoter, an acidic growth promoter, a mineral clay, an enzyme, a metallic substance, a plant-based nutraceutical, or a prebiotic/probiotic.

19. A method for detecting lameness in a pig, the method comprising:
(a) determining a level of at least one biomarker and/or a ratio of two biomarkers in a saliva sample from the pig, wherein the at least one biomarker is C2C, CTX1, CTX2, OC, or P2CP, and the ratio of two biomarkers is OC/CTX1, P2CP/C2C or P2CP/CTX2, and
(b) comparing the level of the at least one biomarker and/or the ratio of two biomarkers in the saliva sample to a lameness negative reference, wherein a change in the level of the at least one biomarker and/or the ratio of two biomarkers in the saliva sample relative to the lameness negative reference indicates that the pig is lame or is predisposed to becoming lame.

20. The method of claim 19, wherein the at least one biomarker is CTX1 or P2CP, and an increased level of CTX1 or P2CP in the saliva sample indicates that the pig is lame or is predisposed to becoming lame.

21. The method of claim 19, wherein the ratio of two biomarkers is P2CP/C2C or P2CP/CTX2, and an increased level of P2CP/C2C or P2CP/CTX2 in the saliva sample indicates that the pig is lame or is predisposed to becoming lame.

22. The method of claim 19, further comprising administering at least one organic mineral to the pig that is lame or is predisposed to becoming lame, wherein administering the at least one organic mineral reduces symptoms of lameness as compared to administering at least one inorganic mineral to a comparable pig afflicted with lameness.
